(19) United States
(12) Patent Application Publication
(10) Pub. No.: US 2025/0213136 A1
(43) Pub. Date: Jul. 3, 2025

(54) METHOD AND SYSTEM FOR EVALUATING AND CLASSIFYING COUGHING ASSOCIATED WITH RESPIRATION

(71) Applicant: THE CATHOLIC UNIVERSITY OF KOREA INDUSTRY-ACADEMIC COOPERATION FOUNDATION, Seoul (KR)

(72) Inventors: Hyunbum KIM, Seoul (KR); Tai Joon AN, Seoul (KR)

(21) Appl. No.: 18/991,981

(22) Filed: Dec. 23, 2024

(30) Foreign Application Priority Data
Dec. 29, 2023 (KR) 10-2023-0197524
May 31, 2024 (KR) 10-2024-0071455
Nov. 11, 2024 (KR) 10-2024-0158833

(51) Int. Cl. A61B 5/08; A61B 5/00; A61B 5/087

(52) U.S. Cl. CPC A61B 5/0823; A61B 5/0803; A61B 5/087; A61B 5/6803; A61B 5/7264; A61B 5/7282; A61B 5/742; A61B 2560/045; A61B 2560/0462; A61B 2562/0204

(57) ABSTRACT

A respiration-associated coughing evaluation system, a respiration-associated coughing evaluation method, and a wearable device that detect the respiration of the measurement object, obtain a respiratory cycle, classify coughing using voice data of the measurement object, determine respiratory information about the measurement object based on the time at which the measurement object starts coughing during the respiratory cycle through the relationship between the respiratory cycle of the measurement object and the start point of coughing, and transmit the respiratory information.

### CROSS-REFERENCE TO RELATED APPLICATION

This application claims priority from Korean Patent Application Nos. 10-2023-0197524, filed on Dec. 29, 2023, 10-2024-0071455, filed on May 31, 2024, and 10-2024-0158833, filed on Nov. 11, 2024, which are hereby incorporated by reference for all purposes as if fully set forth herein.

### BACKGROUND OF THE INVENTION

### Technical Field

The disclosure relates to a respiration-associated coughing evaluation and classification method and a device for obtaining respiratory information about a measurement object through measurement of coughing.

### Description of Related Art

Coughing is one of the common symptoms of the disease, and medical staff and the general public's interest in coughing is increasing after Covid-19. In the treatment and research of coughing, medical doctors judge the form of coughing, such as frequency, pressure, strength, and presence or absence of phlegm, by patient statements or patient-written questionnaires which makes it difficult for the medical doctor to perform accurate diagnosis.

In order to objectify a patient's coughing, there is a discussion about measuring the frequency of coughing through a mask, the pressure and speed of the air from coughing (see non-patent literature 1).

### BRIEF SUMMARY

Embodiments of the disclosure provide a respiration-associated coughing evaluation system, a respiration-associated coughing evaluation method, and a wearable device that obtain coughing information related to a respiratory state through a device rather than a patient and provides information about a respiratory system based on the obtained coughing information.

In an aspect, a respiration-associated coughing evaluation system according to an embodiment, comprises a wearable device that detects the respiration of the measurement object, obtains a respiratory cycle, classifies coughing using voice data of the measurement object, determines respiratory information about the measurement object based on the time at which the measurement object starts coughing during the respiratory cycle through the relationship between the respiratory cycle of the measurement object and the start point of coughing, and transmits the respiratory information, and a terminal that receives and displays the respiratory information transmitted from the wearable device, and transmits a control signal to the wearable device.

In this case, the respiratory cycle is divided into an inspiratory section and an expiratory section. When determining the respiratory information about the measurement object, the wearable device determines that the measurement object starts coughing before completing the inspiratory section of the respiratory cycle, the measurement object starts coughing with the start of the expiratory section after completing the inspiratory section of the respiratory cycle, or the measurement object starts coughing in the middle of the inspiratory section of the respiratory cycle or the measurement object starts coughing at the end of the expiratory section of the respiratory cycle.

In another aspect, a wearable device comprises a main body including a controller detecting respiration of a measurement object to obtain a respiratory cycle, dividing coughing using voice data of the measurement object, and determining respiratory information about the measurement object based on a time when the measurement object starts coughing in the respiratory cycle through a relationship between the respiratory cycle of the measurement object and a start point of the coughing, and a communication unit transmitting the respiratory information, a band connected to the main body and detecting the respiration of the measurement object, and a neck connection microphone detecting a sound made in or around a neck of the measurement object to obtain the voice data of the measurement object.

In this case, the respiratory cycle is divided into an inspiratory section and an expiratory section. When determining the respiratory information about the measurement object, the controller of the main body determines that the measurement object starts coughing before completing the inspiratory section of the respiratory cycle, the measurement object starts coughing with the start of the expiratory section after completing the inspiratory section of the respiratory cycle, or the measurement object starts coughing in the middle of the inspiratory section of the respiratory cycle or the measurement object starts coughing at the end of the expiratory section of the respiratory cycle.

In another aspect, a respiration-associated coughing evaluation system according to another embodiment comprises a wearable device transmitting a respiratory cycle and voice data of a measurement object obtained by detecting respiration of the measurement object, a server dividing coughing using the voice data of the measurement object, determining respiratory information about the measurement object based on a time when the measurement object starts coughing in the respiratory cycle through a relationship between the respiratory cycle of the measurement object and a start point of the coughing, and transmitting the respiratory information, and a terminal receiving and displaying the respiratory information transmitted from the server.

In this case, the respiratory cycle is divided into an inspiratory section and an expiratory section. When determining the respiratory information about the measurement object, the server determines that the measurement object starts coughing before completing the inspiratory section of the respiratory cycle, the measurement object starts coughing with the start of the expiratory section after completing the inspiratory section of the respiratory cycle, or the measurement object starts coughing in the middle of the inspiratory section of the respiratory cycle or the measurement object starts coughing at the end of the expiratory section of the respiratory cycle.

In another aspect, a respiration-associated coughing evaluation method according to another embodiment comprises determining respiratory information about a measurement object based on a time when the measurement object starts coughing in an inspiratory section and an expiratory section and storing or transmitting the respiratory information.

In this case, the respiratory cycle is divided into an inspiratory section and an expiratory section. When determining the respiratory information about the measurement object, determining the respiratory information determines that the measurement object starts coughing before completing the inspiratory section of the respiratory cycle, the measurement object starts coughing with the start of the expiratory section after completing the inspiratory section of the respiratory cycle, or the measurement object starts coughing in the middle of the inspiratory section of the respiratory cycle or the measurement object starts coughing at the end of the expiratory section of the respiratory cycle.

A respiration-associated coughing evaluation system, a respiration-associated coughing evaluation method, and a wearable device may obtain coughing information related to a respiratory state through a device rather than a patient, and provide information about a respiratory system based on the obtained coughing information.

### BRIEF DESCRIPTION OF DRAWINGS

The above and other aspects, features, and advantages of the disclosure will be more clearly understood from the following detailed description, taken in conjunction with the accompanying drawings, in which:

FIG. 1 is a conceptual view illustrating a respiration-associated coughing evaluation system according to an embodiment of the disclosure;

FIG. 2 illustrates an example of a wearable device according to another embodiment of the disclosure;

FIG. 3 illustrates another example of the wearable device of FIG. 2;

FIG. 4 is a block diagram illustrating a main body of FIGS. 2 and 3;

FIG. 5A illustrates an air flow rate and a section divided over time according to an embodiment of the disclosure;

FIG. 5B illustrates an example of a normal respiratory cycle of an observed object;

FIG. 5C illustrates an example of a respiratory cycle of an observed object during inspiratory hypersensitive coughing;

FIG. 5D illustrates an example of a respiratory cycle of an observed object during inspiratory spontaneous coughing;

FIG. 5E illustrates an example of a respiratory cycle of an observed object during expiratory hypersensitive coughing;

FIG. 5F illustrates an example of a respiratory cycle of an observed object during irritating coughing;

FIG. 6 is a plan view illustrating a neck connection microphone of FIGS. 2 and 3;

FIG. 7 is an external cross-sectional view taken along line AA′ of FIG. 6;

FIG. 8A is a front view illustrating a mask added to the wearable device of FIGS. 2 and 3;

FIG. 8B is a front view illustrating another example of a mask added to the wearable device of FIGS. 2 and 3;

FIG. 9A is an example of flow rate information about a normal breathing sound obtained through the mask of FIGS. 8B and 8B;

FIG. 9B is an example of flow rate information about a coughing sound obtained through the mask of FIGS. 8A and 8B;

FIG. 9C is an example of flow rate information about a conversation sound obtained through the mask of FIGS. 8A and 8B;

FIG. 10 is a conceptual view illustrating a respiration-associated coughing evaluation system according to another embodiment of the disclosure; and

FIG. 11 is a flowchart illustrating a respiration-associated coughing method according to another embodiment of the disclosure.

### DETAILED DESCRIPTION OF THE PREFERRED EMBODIMENTS OF THE INVENTION

Hereinafter, embodiments of the disclosure are described in detail with reference to the accompanying drawings. In assigning reference numerals to components of each drawing, the same components may be assigned the same numerals even when they are shown on different drawings. When determined to make the subject matter of the disclosure unclear, the detailed of the known art or functions may be skipped. The terms “comprises” and/or “comprising,” “has” and/or “having,” or “includes” and/or “including” when used in this specification specify the presence of stated features, regions, integers, steps, operations, elements, and/or components, but do not preclude the presence or addition of one or more other features, regions, integers, steps, operations, elements, components, and/or groups thereof. As used herein, the singular forms “a”, “an” and “the” are intended to include the plural forms as well, unless the context clearly indicates otherwise.

Such denotations as “first,” “second,” “A,” “B,” “(a),” and “(b),” may be used in describing the components of the disclosure. These denotations are provided merely to distinguish a component from another, and the essence, order, or number of the components are not limited by the denotations.

In describing the positional relationship between components, when two or more components are described as “connected”, “coupled” or “linked”, the two or more components may be directly “connected”, “coupled” or “linked”, or another component may intervene. Here, the other component may be included in one or more of the two or more components that are “connected”, “coupled” or “linked” to each other.

When such terms as, e.g., “after”, “next to”, “after”, and “before”, are used to describe the temporal flow relationship related to components, operation methods, and fabricating methods, it may include a non-continuous relationship unless the term “immediately” or “directly” is used.

Meanwhile, if a numerical value or its corresponding information (e.g., reference value etc.) is mentioned for a component, it may be interpreted that the numerical value or its corresponding information includes a margin of error that may be caused by various factors (e.g., process factors, internal or external shocks, noise, etc.), even if it is not explicitly stated otherwise.

Embodiments are described below with reference to the drawings, but the disclosure is not limited thereto.

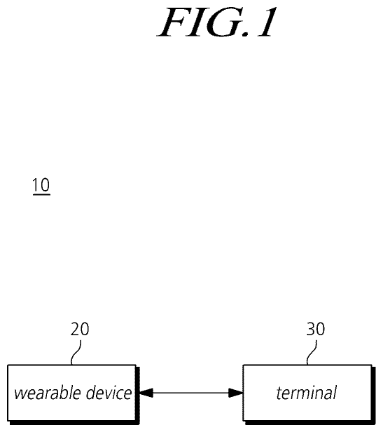

FIG. 1 is a conceptual view illustrating a respiration-associated coughing evaluation system according to an embodiment of the disclosure.

Referring to FIG. 1, a respiration-associated coughing evaluation system 10, according to an embodiment, includes a wearable device 20 that detects the respiration of the measurement object, obtains a respiratory cycle, classifies coughing using voice data of the measurement object, determines respiratory information about the measurement object based on the time at which the measurement object starts coughing during the respiratory cycle through the relationship between the respiratory cycle of the measurement object and the start point of coughing, and transmits the respiratory information, and a terminal 30 that receives and displays the respiratory information transmitted from the wearable device 20, and transmits a control signal to the wearable device 20.

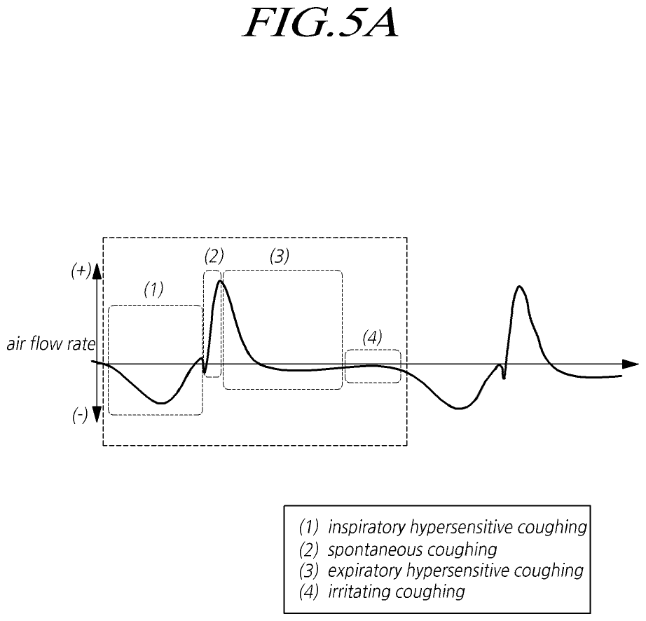

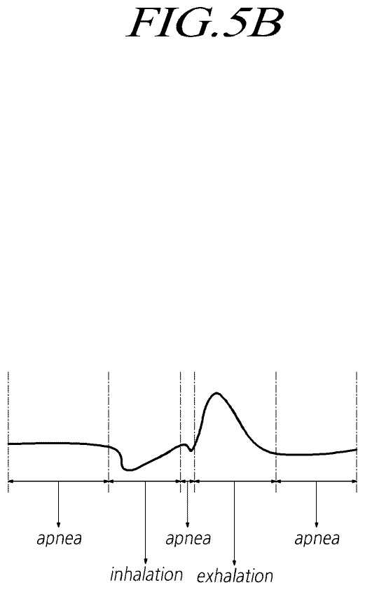

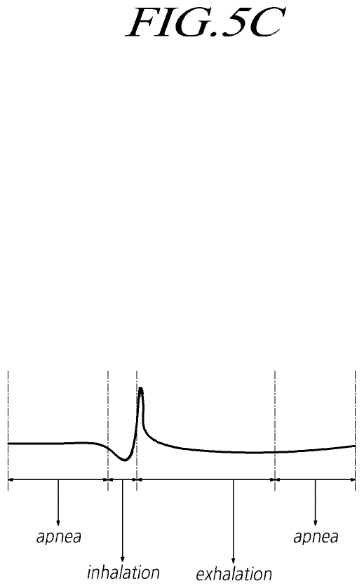

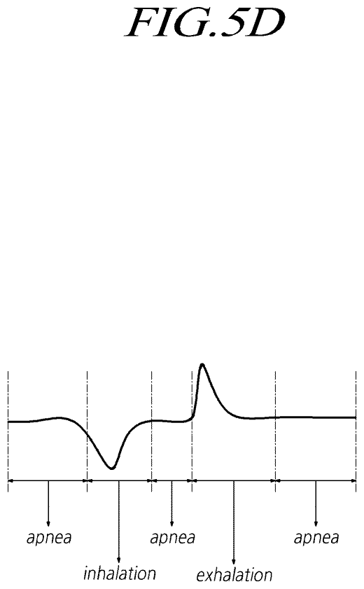

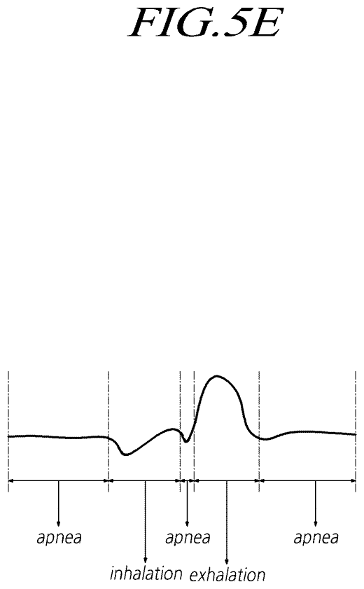

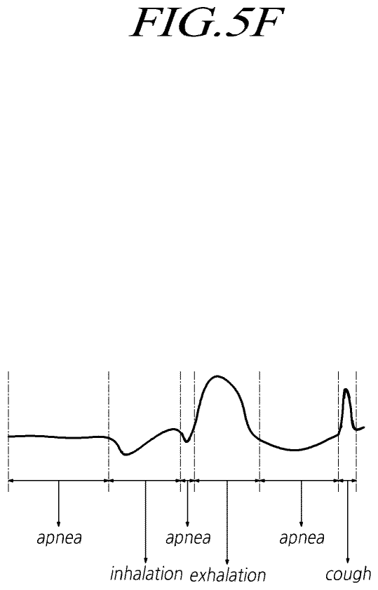

As described below with reference to FIG. 5, the respiratory cycle may be divided into an inspiratory section and an expiratory section, but the disclosure is not limited thereto.

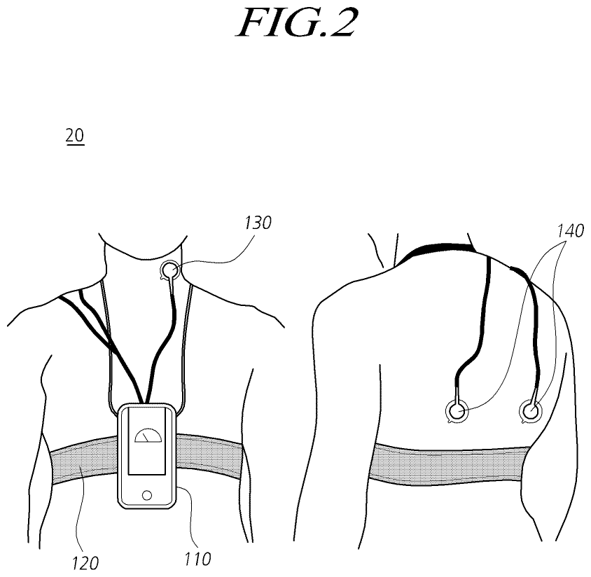

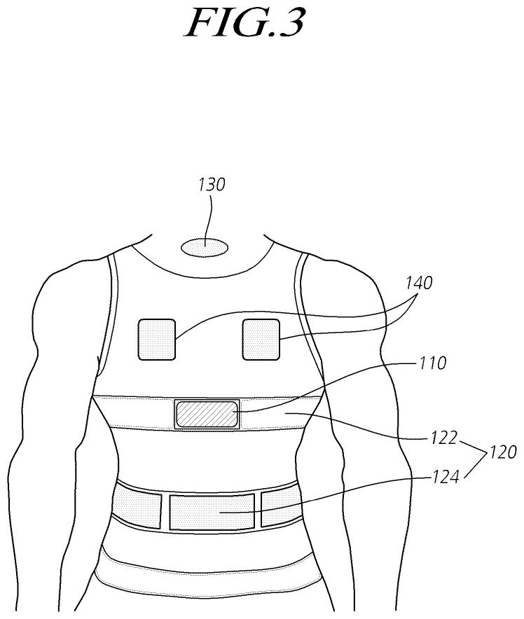

When determining the respiratory information about the measurement object, the wearable device 20 determines that the measurement object starts coughing before completing the inspiratory section of the respiratory cycle, the measurement object starts coughing with the start of the expiratory section after completing the inspiratory section of the respiratory cycle, or the measurement object starts coughing in the middle of the inspiratory section of the respiratory cycle or the measurement object starts coughing at the end of the expiratory section of the respiratory cycle. The wearable device 20 is described below with reference to FIGS. 2 and 3.

As shown in FIG. 2, the wearable device 20 may be in a wearable form such as a vest or may be an attachment tape. In other words, the wearable device 20 may correspond to a wearable device that may be worn by the measurement object, such as a sports bra, sportswear, and diagnostic wear used in hospitals, as shown in FIG. 3.

For example, the wearable device 20 may record and analyze coughing and breathing sounds by mounting a small microphone and various sensors on a wearable vest that may be worn under daily clothing (including underwear). When the patient wears the wearable vest, he may automatically measure and record coughing during his routines 24 hours a day. Thereafter, the start point of coughing may be classified into four types described below according to the respiratory cycle and a result table may be created.

The wearable device 20 determines that if the measurement object starts coughing before the inspiratory section of the respiratory cycle is completed, it is a first disease, determines that if the measurement object starts coughing with the start of the expiratory section after completing the inspiratory section of the respiratory cycle, it is a second disease, determines that if the measurement object starts coughing in the middle of the expiratory section of the respiratory cycle, it is a third disease, and determines that if the measurement object starts coughing at the end of the expiratory section of the respiratory cycle, it is a fourth disease.

When determining the respiratory information about the measurement object, the wearable device 20 may determine the respiratory information by further considering at least one of the frequency of coughing per predetermined unit time, the ratio of the inspiratory amount to the amount of air exhaled during coughing, the peak pressure of coughing, and the ratio of coughing having a predetermined intensity or more to the total coughing.

The terminal 30 may be any one of a mobile phone, a smartphone, a general computer, a laptop computer, a digital broadcasting terminal, a personal digital assistant (PDA), a portable multimedia player (PMP), a navigation, a slate PC, a tablet PC, an Ultrabook, a wearable device (e.g., a watch-type terminal (smartwatch), a glasses-type terminal (smart glass), and a head mounted display (HMD)).

In various embodiments of the disclosure, the respiratory information may include information about a disease, symptom, and illness related to the respiratory system. For example, the respiratory information may include information about the first to fourth diseases.

FIG. 2 illustrates an example of a wearable device according to another embodiment of the disclosure. FIG. 3 illustrates another example of the wearable device of FIG. 2.

Referring to FIG. 2, the wearable device 20 according to another embodiment may be a kind of information acquisition device. The wearable device 20 includes a main body 110, a band 120, a neck connection microphone 130, and a chest microphone 140. The chest microphone includes a chest microphone or a back microphone.

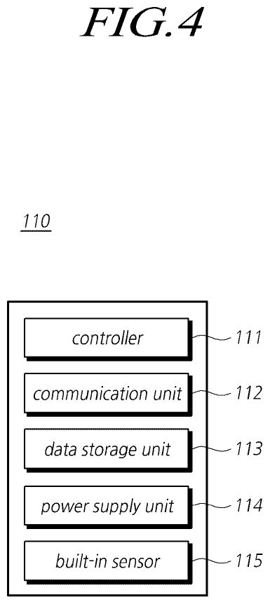

FIG. 4 is a block diagram illustrating a main body of FIGS. 2 and 3.

Referring to FIG. 4, the main body 110 may include a controller 111, a communication unit 112, a power source or power supply unit 114, a data storage unit 113, and a built-in sensor 115. The main body 100 may further include a synchronization unit as needed.

The controller 111 may include one or more processors. The controller 111 may determine the respiratory information about the measurement object based on at which point in the respiratory cycle the measurement object starts coughing. The controller 111 may obtain the respiratory cycle based on the respiration of the measurement object detected by the band and classify the coughing using the voice data of the measurement object.

Specifically, the controller 111 determines that if the measurement object starts coughing before the inspiratory section of the respiratory cycle is completed, it is a first disease, determines that if the measurement object starts coughing with the start of the expiratory section after completing the inspiratory section of the respiratory cycle, it is a second disease, determines that if the measurement object starts coughing in the middle of the expiratory section of the respiratory cycle, it is a third disease, and determines that if the measurement object starts coughing at the end of the expiratory section of the respiratory cycle, it is a fourth disease.

When determining the respiratory information about the measurement object, the controller 111 may determine the respiratory information by further considering at least one of the frequency of coughing per predetermined unit time, the ratio of the inspiratory amount to the amount of air exhaled during coughing, the peak pressure of coughing, and the ratio of coughing having a predetermined intensity or more to the total coughing.

The communication unit 112 may communicate with the terminal 30 or a server wiredly or wirelessly through a wireless network or a wired network. The communication unit 112 may transmit the respiratory information and receive control information.

The built-in sensor 115 may include one or more sensors, e.g., a position change detection sensor and/or a pressure sensor. Alternatively, a gravity sensor for posture detection may be included.

The power supply unit 114 serves to supply power to other components and may include a battery.

The data storage unit 113 may be referred to as a storage unit, and may record respiratory information about a measurement target (patient).

The synchronization unit not shown may provide a criterion for the relationship between coughing, expiratory air, and inhalation from a temporal point of view.

The band 120 may be referred to as a chest band, a thorax band, an abdominal band, or the like, and fixes the main body 110 to a part of the body. For example, the band 120 may include only the chest band as illustrated in FIG. 2, or the chest band 122 that detects respiration through the chest and the abdominal band 124 that detects respiration through the abdomen as illustrated in FIG. 3. The band 120 may be connected to the main body 110 and may detect respiration of the measurement object. When the chest band 122 mainly detects the respiration of the measurement object and the abdominal band 124 is provided separately as shown in FIG. 3, the abdominal band 124 may assist in detecting the respiration of the measurement object.

The band 120 may be embedded with a tension sensor including a fixing portion (not shown) and a variable portion (not shown). The respiration of the measurement object may be detected by utilizing the degree of stretching of the variable portion of this tension sensor.

As described above, the controller 111 of the main body 110 may obtain the respiratory cycle based on the respiration of the measurement object detected by the band 120.

The band 120 makes the information acquisition device into the wearable device 20 or a device, and does not necessarily have the shape of a band, and as described above, the wearable device 20 may be in a wearable form such as a vest or an attachment tape as shown in FIG. 2, or may correspond to a wearable device such as a sports bra or sportswear as shown in FIG. 3.

As described with reference to FIGS. 2 and 3, the microphone may include only the neck connection microphone 130 or may include the chest microphone 140 together with the neck connection microphone. The chest microphone 140 may include a back microphone as shown in FIG. 2 and/or a chest microphone as shown in FIG. 3, attached to the front and back of both lungs of the measurement object.

The neck connection microphone 130 may detect a subglottic sound. In other words, the neck connection microphone 130 may detect a sound made in or around the neck of the measurement object to obtain voice data of the measurement object.

The controller 111 of the main body 110 may distinguish coughing using the voice data of the measurement target. As a result, the controller 111 of the main body 110 may determine respiratory information about the measurement object based on the time point at which the measurement object starts coughing during the respiratory cycle.

The controller 111 of the main body 110 may use a general noise removal algorithm or use a trained artificial intelligence on coughing and other noise using voice data recorded by the microphone 130 and 140.

The chest microphone 140 may detect the presence or absence of a lung disease, a crackle, and a wheezing.

In other words, the chest microphone 140 detects sound such as from a lung, and the neck connection microphone 130 detects sound made in or around the neck.

In various embodiments of the disclosure, the neck connection microphone 130 may remove noise other than coughing.

In various embodiments of the disclosure, the chest microphone 140 may be referred to as a lung microphone or an auxiliary microphone, and the neck connection microphone 130 may be referred to as a subglottic microphone or a main microphone.

Further, these microphones 130 and 140 may be attached in patches, bands, or a wearable device and thus may not be visible from the outside. Each of the microphones 130 and 140 may be connected to the main body 110 wiredly or wirelessly (including Wi-Fi and Bluetooth). There is not necessarily one chest microphone 140 on each side, but there is one or more chest microphones on one side, so that lung sound analysis may be performed more accurately. Further, if necessary, lung sounds may be heard from the front of the chest.

Lung sounds and sounds from the throat obtained through the chest microphone 140 and the neck connection microphone 130 may be referred to for identifying the cause of coughing using artificial intelligence or the like. For example, in the section (1) of FIG. 5, if a crackle is heard from the lung sound of a patient who coughed, it may be diagnosed as pneumonia.

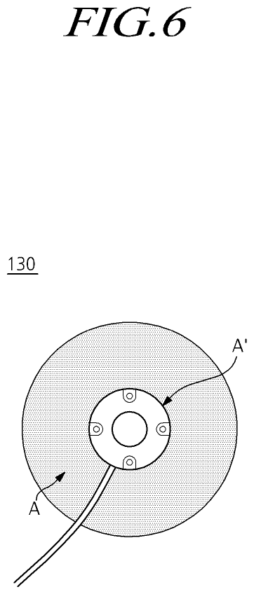

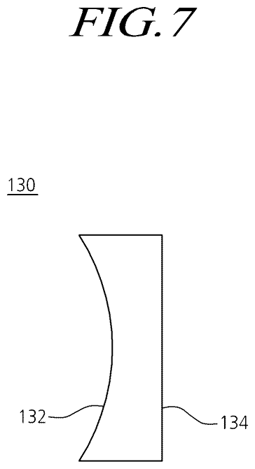

FIG. 6 is a plan view illustrating a neck connection microphone of FIGS. 2 and 3. FIG. 7 is an external cross-sectional view taken along line AA′ of FIG. 6.

Referring to FIGS. 6 and 7, these microphones 130 or 140 may have a shape in which the center of the front surface is concave as illustrated in FIGS. 6 and 7, but are not limited thereto. Since the center portion of the front surface of the microphones 130 and 140 has a concave shape, sound made in or around the neck or neck may be enlarged.

As another example, these microphones 130 or 140 may have a concave central portion of the front and/or rear surface. When the microphones 130 or 140 have concave center portions in the front/rear surface, the curvatures of both the sides may be the same or different. The outer portion of the microphone 130 or 140 may have an oval shape to be suitable for the shape of the neck.

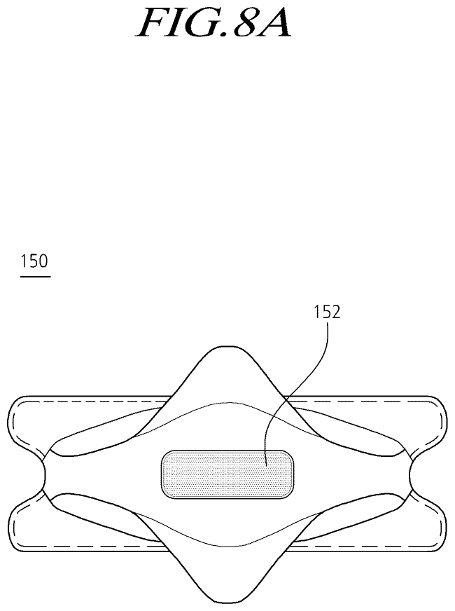

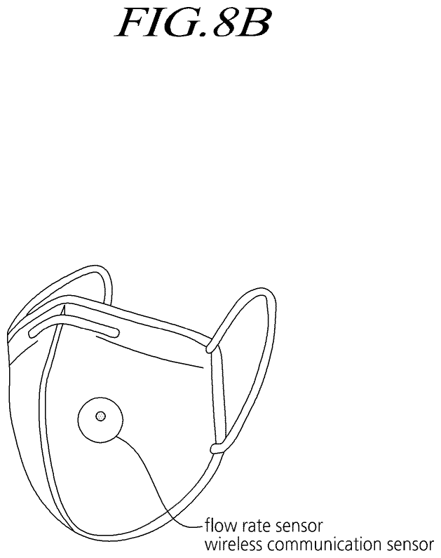

FIG. 8A is a front view illustrating an example of a mask added to the wearable device of FIGS. 2 and 3. FIG. 8B is a front view illustrating another example of a mask added to the wearable device of FIGS. 2 and 3.

As shown in FIGS. 8A and 8B, the wearable device 20 of FIGS. 2 and 3 may further include a mask 150 including a flow rate/flow velocity sensor 152 that is electrically or communicatively connected to the main body 110 and measures the flow rate or flow velocity of respiration on the inner surface.

The wearable device 20 of FIGS. 2 and 3 may include a mask 150 instead of, or in addition to, the microphones 130 and 140.

The mask 150 is provided with a flow rate/flow velocity sensor 152 (e.g., a pressure sensor, etc.), so that the frequency, pressure, or speed of coughing may be measured. The mask 150 may include a communication unit (e.g., a Bluetooth device, a Wi-Fi device) capable of communicating with the main body 110 or other devices. The communication unit (not shown) may be provided in the wearable device 20 of FIGS. 2 and 3 separately from the mask 150. The mask 150 may include one or more fans.

The wearable device 20 of FIGS. 2 and 3 may train artificial intelligence with the flow rate information (the amount of air exhaled when coughing) obtained from the mask 150, ultimately obtaining the flow rate information about coughing by attaching only a sensor (e.g., the neck connection microphone 130) to the neck without a mask. As described above, coughing is a process in which the subglottic pressure rises after the glottis is closed and then the glottis opens suddenly, releasing air quickly and hard. The subglottic pressure and the amount of air passing through the glottis are very important. Therefore, it is possible to measure the amount and pressure of air of coughing through the attachment neck connection microphone 130 on the glottis portion even without wearing the mask 150 by measuring the strength and amount exhaled from the mouth using the flow rate sensor 152 in the mask 150 while simultaneously attaching the neck connection microphone 130 to the glottis portion to train the artificial intelligence at the same time with recording.

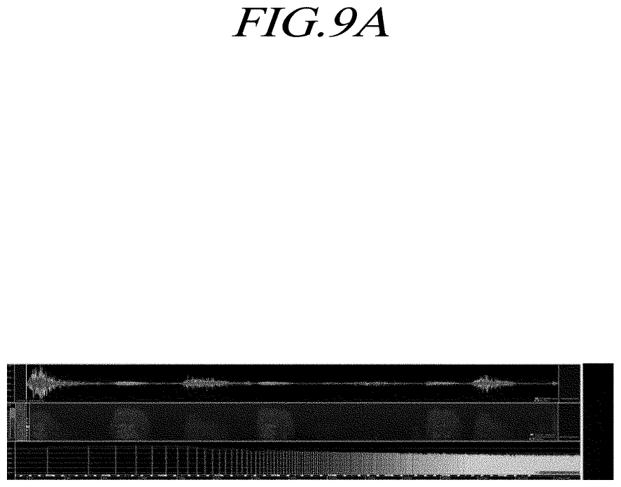

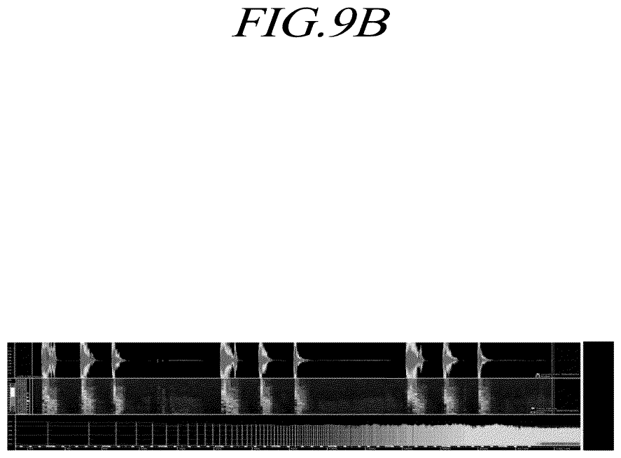

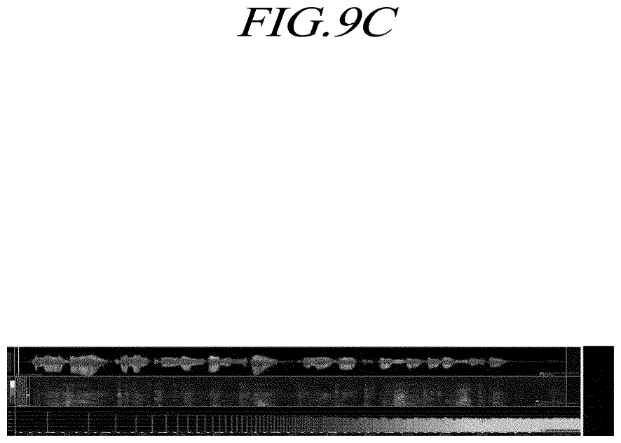

FIG. 9A is an example of flow rate information about a normal breathing sound obtained through the mask of FIGS. 8B and 8B. FIG. 9B is an example of flow rate information about a coughing sound obtained through the mask of FIGS. 8A and 8B. FIG. 9C is an example of flow rate information about a conversation sound obtained through the mask of FIGS. 8A and 8B.

By training the artificial intelligence using flow rate information about the normal respiration sound and coughing sound and conversation sound of FIGS. 9A to 9C and using the trained artificial intelligence, it is possible to predict the amount and pressure of air of coughing through the attachment neck connection microphone 130 on the glottis portion even without the mask 150. Therefore, in various embodiments of the disclosure, the wearable device 20 or the information acquisition device of FIGS. 2 and 3 may be equipped with an artificial intelligence function to predict the amount and pressure of air of coughing only through the neck connection microphone 130 attached to the glottis portion.

However, the size and shape of the glottis and the thickness of the skin fat vary greatly from person to person. Thus, after massive training, the sound and amount may be measured using the neck connection microphone 130 when first worn, and fine tuning may be individually performed. In various embodiments of the disclosure, a patient's respiration may be detected (measured) through at least one of a microphone connected to the neck, a microphone attached to the back, a chest band, an abdominal band, and a mask among the configurations included in the wearable device 20 or the information acquisition device of FIGS. 2 and 3.

In various embodiments of the disclosure, a patient's coughing may be detected (measured) through at least one of a microphone connected to the neck, a microphone attached to the back, a chest band, an abdominal band, and a mask among the configurations included in the wearable device 20 or the information acquisition device of FIGS. 2 and 3.

In various embodiments of the disclosure, the wearable device 20 or the information acquisition device of FIGS. 2 and 3 may be worn by the target patient to continuously detect information about coughing for a given time (e.g., 24 hours). Information measured by each component may be recorded by the data storage unit 113.

FIG. 5A illustrates an air flow rate and a section divided over time according to an embodiment of the disclosure.

Cough is the discharge of air inside the lungs accompanied by an increase in subglottic pressure, and coughing may be affected by respiratory conditions.

Respiration may be divided into an inspiratory section for inhaling and an expiratory section for exhaling from a temporal point of view. There may be some apnea times between the inhalation and the exhalation, which may be classified as being included in at least one of the inspiratory section and the expiratory section.

In FIG. 5A, a section in which the air flow rate is negative (−) is the inspiratory section, and a section in which the air flow rate is positive (+) is the inspiratory section.

In FIG. 5A, section (1) represents the inspiratory section. In FIG. 5A, section (2) represents the start expiratory section. In FIG. 5A, section (3) represents the mid expiratory section. In FIG. 5A, section (4) represents the end expiratory section.

FIG. 5B illustrates an example of a normal respiratory cycle of an observed object.

When the observed object breathes normally, e.g., as illustrated in FIG. 5B, a general normal respiratory cycle is shown. An apnea section may be present before the inspiratory section of section (1), and an apnea section may be present between inspiratory section (1) and start expiratory section (2). If the inspiratory section (2) to (4) ends, another apnea section starts. Thereafter, the inspiratory section of section (1) starts.

When the target patient starts coughing in section (1), i.e., before the inspiratory section is completed, the target patient may be determined as having a first disease. Coughing in section (1) may be referred to as inspiratory coughing or an inspiratory hypersensitive coughing, or inhalation reduced coughing.

FIG. 5C illustrates an example of a respiratory cycle of an observed object during inspiratory hypersensitive coughing.

As shown in FIG. 5C, the air flow rate in section (1) is reduced in the respiratory cycle of the observed object during the inspiratory hypersensitive coughing. Therefore, the inspiratory hypersensitive coughing may be referred to as inhalation reduced coughing.

Inspiratory hypersensitive coughing is coughing in the middle of inhaling, and its common cause may be aspiration. The first disease corresponding thereto may include, e.g., at least one of pneumonia, tuberculosis, and aspiration.

FIG. 5D illustrates an example of a respiratory cycle of an observed object during inspiratory spontaneous coughing.

As shown in FIG. 5D, in the respiratory cycle of the observed object during the spontaneous coughing, the air flow rate in section (2) is increased. Therefore, spontaneous coughing may also be referred to as inhalation increased coughing.

In FIG. 5D, section (2) represents the start expiratory section. (2) In the section, i.e., when the coughing of the target patient starts with the start of the expiratory air after the inspiratory is completed, it may be determined that the target patient has the second disease. Coughing in section (2) may be referred to as spontaneous coughing. Spontaneous coughing starts after completing the inhalation, and is coughing when one inhales as much as or more than usual to make coughing, and its common cause is pharyngeal nerve hypersensitivity and persistent pharyngeal irritation. The second disease may correspond to, e.g., habitual coughing or upper respiratory coughing syndrome.

FIG. 5E illustrates an example of a respiratory cycle of an observed object during expiratory hypersensitive coughing.

As shown in FIG. 5E, the air flow rate in section (3) is increased in the respiratory cycle of the observed object during the expiratory hypersensitive coughing.

In FIG. 5E, section (3) represents the mid expiratory section. When the target patient starts coughing in section (3), the target patient may be determined as having a third disease. Coughing in section (3) may be referred to as expiratory coughing or an expiratory hypersensitive coughing, or exhalation increased coughing. Expiratory hypersensitive coughing is coughing that suddenly starts in the middle of exhalation, and its most common cause is inflammation or narrowing of the trachea and main bronchus. The third disease may correspond to at least one of, e.g., an upper respiratory tract infection, asthma, and chronic obstructive pulmonary disease.

FIG. 5F illustrates an example of a respiratory cycle of an observed object during irritating coughing.

As shown in FIG. 5E, the air flow rate in section (4) is increased in the respiratory cycle of the observed object during the irritating coughing. Coughing in section (4) may start without a change in the air flow rate.

In FIG. 5F, section (4) represents the end expiratory section. When the target patient starts coughing in section (4), the target patient may be determined as having a fourth disease. Coughing in section (4) may be referred to as hypersensitive or irritating coughing. Coughing in section (4) may start without a change in the air flow rate. Irritating coughing is coughing that occurs in the apnea section between a respiration and another respiration, and false coughing or coughing due to psychological cause may show such a pattern. The fourth disease may correspond to at least one of, e.g., neurotic coughing, reflux esophagitis, and tic symptoms.

Overall, this classification is not simply the number or intensity of coughing, but it may affect treatment plans as a classification that may directly affect treatment.

According to various embodiments of the disclosure, a patient wears the wearable device 20 or the information acquisition device of FIGS. 2 and 3 during his/her daily life, and the wearable device 20 of FIGS. 2 and 3 may help doctors in medical treatment by transferring information about the respiratory system to the terminal 30.

According to various embodiments of the disclosure, information about the conditions of the patient's respiratory system may be provided based on the timing when the patient coughs by identifying the respiratory cycle of the observed object of FIGS. 5C to 5F.

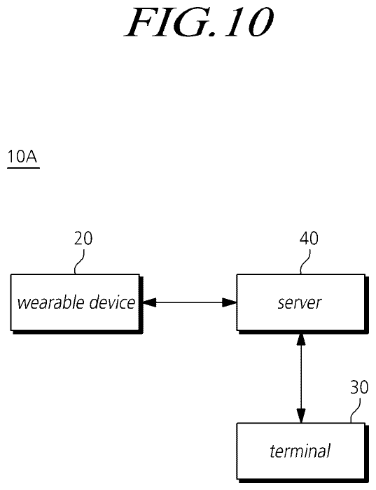

FIG. 10 is a conceptual view illustrating a respiration-associated coughing evaluation system according to another embodiment of the disclosure.

Referring to FIG. 10, a respiration-associated coughing evaluation system 10A, according to another embodiment, includes a wearable device 20 that transmits voice data of a measurement object and a respiratory cycle obtained by detecting the respiration of the measurement object, a server 40 that classifies coughing using voice data of the measurement object, determines respiratory information about the measurement object based on the time at which the measurement object starts coughing during the respiratory cycle through the relationship between the respiratory cycle of the measurement object and the start point of coughing, and transmits the respiratory information, and a terminal 30 that receives and displays the respiratory information transmitted from the server 40.

The respiration-associated coughing evaluation system 10A according to another embodiment described with reference to FIG. 10 is substantially the same as the respiration-associated coughing evaluation system 10 according to an embodiment described with reference to FIG. 1 but only differs in that the wearable device 20 transmits, to the server 40, the voice data of the measurement object and the respiratory cycle of the measurement object obtained through the band 120 and the microphone 130 and/or 140, and the server 40 classifies coughing using the voice data of the measurement object, determines the respiratory information about the measurement object based on the time when the measurement object starts coughing during the respiratory cycle through the relationship between the respiratory cycle of the measurement object and the start point of coughing, and transmits the respiratory information. In other words, the wearable device 20 may transmit the voice data of the measurement object and the respiratory cycle of the measurement object obtained through the band 120 and the microphone 130 and/or 140, and it may be the server 40 that determines the respiratory information about the measurement object using the start point of coughing and the respiratory cycle.

The terminal 30 may receive respiratory information by connecting to the server 40 wiredly or wirelessly through a wireless network or a wired network. The medical staff may finally treat the patient based on the respiratory information provided through the terminal 30.

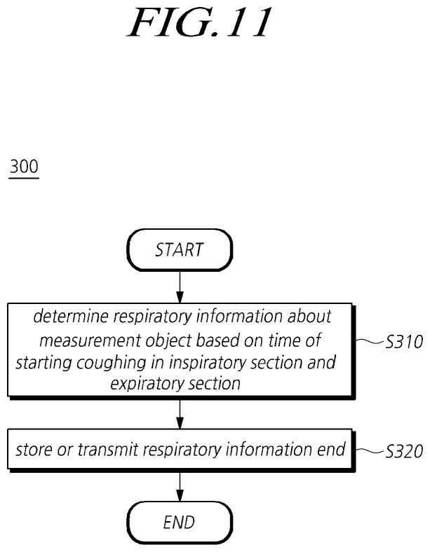

FIG. 11 is a flowchart illustrating a respiration-associated coughing method according to another embodiment of the disclosure.

Referring to FIG. 11, a respiration-associated coughing evaluation method 30 according to another embodiment includes a step S310 of determining respiratory information about a measurement object based on a time when the measurement object starts coughing in an inspiratory section and an expiratory section and a step S320 of storing or transmitting the respiratory information.

In this case, the respiratory cycle is divided into an inspiratory section and an expiratory section. When determining the respiratory information about the measurement object, determining the respiratory information determines that the measurement object starts coughing before completing the inspiratory section of the respiratory cycle, the measurement object starts coughing with the start of the expiratory section after completing the inspiratory section of the respiratory cycle, or the measurement object starts coughing in the middle of the inspiratory section of the respiratory cycle or the measurement object starts coughing at the end of the expiratory section of the respiratory cycle.

The respiration-associated coughing evaluation method 30 determines the respiratory information about the measurement object based on the time when the measurement object starts coughing in the inspiratory section and the expiratory section (S310).

The respiratory information may be classified into four as follows, but is not limited thereto. For example, if the measurement object starts coughing before the inspiratory section is completed, it may be determined as a first disease, if the measurement object starts coughing with the start of the expiratory section after completing the inspiratory section, it may be determined as a second disease, if the measurement object starts coughing in the middle of the expiratory section, it may be determined as a third disease, and if the measurement object starts coughing at the end of the expiratory section, it may be determined as a fourth disease.

Next, the respiration-associated coughing evaluation method 30 stores the determined respiratory information as data or transmits it to another device such as a server (S320).

The respiration-associated coughing evaluation method 30 may utilize other measurement information. For example, the respiratory information may be determined by further considering at least one of the frequency of coughing per predetermined unit time, the ratio of the inspiratory amount to the amount of air exhaled during coughing, the peak pressure of coughing, and the ratio of coughing (expulsive event) having a predetermined intensity or more to the total coughing.

The frequency of coughing per predetermined unit time may be determined, e.g., two or more times per minute (short-term based), 15 times or more in one hour (medium-term based), and 50 times or more in 24 hours (long-term based). Here, the frequency of coughing may be defined as the frequency of coughing having a predetermined strength or more or a value obtained by multiplying or dividing the frequency by a predetermined value.

The ratio of the amount of air inhaled to the amount of air exhaled during coughing may be determined, e.g., by whether the amount of air exhaled during coughing is 120% of the amount inhaled or more.

The ratio of the coughing having the predetermined strength or more to the total coughing may be determined by, e.g., whether it is 30% or more or 50% or more.

As described above, in the respiration-associated coughing evaluation method 30, lung sounds and sounds from the throat obtained through the chest microphone 140 and the neck connection microphone 130 may be referred to for identifying the cause of coughing using artificial intelligence or the like. For example, in the section (1) of FIG. 2, if a crackle is heard from the lung sound of a patient who coughed, it may be diagnosed as pneumonia.

The above-described respiration-associated coughing evaluation method 30 may obtain coughing information related to a respiratory state through a device rather than a patient, and provide information about a respiratory system based on the obtained coughing information.

In the description of the disclosure, the respiration-associated coughing evaluation system and the respiration-associated coughing evaluation method, and the wearable device are divided, but they are common in that respiratory information is provided using the respiratory cycle and the start point of coughing. Therefore, they should be comprehensively understood in the disclosure, and the content of one part may be used as the content of another part.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “unit” may include a unit implemented in hardware, software, or firmware, and may interchangeably be used with other terms, for example, “logic,” “logic block,” “part,” or “circuitry”. A unit may be a single integral component, or a minimum unit or part thereof, adapted to perform one or more functions. For example, according to an embodiment, the unit may be implemented in a form of an application-specific integrated circuit (ASIC).

Various embodiments as set forth herein may be implemented as software (e.g., the program) including one or more instructions that are stored in a storage medium (e.g., internal memory or external memory) that is readable by a machine (e.g., the display device). For example, a processor of the machine (e.g., the display device) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The storage medium readable by the machine may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. Some of the plurality of entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

Preferred embodiments of the disclosure have been described above. The above-described embodiments are merely examples, and it will be appreciated by one of ordinary skill in the art various changes may be made thereto without departing from the scope of the disclosure. Therefore, the disclosed embodiments should be considered from an illustrative, rather than a limiting, point of view. The scope of various embodiments of the disclosure is indicated in the claims rather than in the above-described description. All differences within the equivalent range should be construed as being included in the disclosure.

1. A respiration-associated coughing evaluation, comprising:
a wearable device detecting respiration of a measurement object to obtain a respiratory cycle, dividing coughing using voice data of the measurement object, determining respiratory information about the measurement object based on a time when the measurement object starts coughing in the respiratory cycle through a relationship between the respiratory cycle of the measurement object and a start point of the coughing, and transmitting the respiratory information; and
a terminal receiving and displaying the respiratory information transmitted from the wearable device and transmitting a cell source to the wearable device, wherein the respiratory cycle is divided into an inspiratory section and an expiratory section, and wherein when determining the respiratory information about the measurement object, the wearable device determines whether the measurement object starts coughing before completing the inspiratory section of the respiratory cycle, whether the measurement object starts coughing with a start of the expiratory section after completing the inspiratory section of the respiratory cycle, whether the measurement object starts coughing in a middle of the expiratory section of the respiratory cycle, and whether the measurement object starts coughing at an end of the expiratory section of the respiratory cycle.

2. The respiration-associated coughing evaluation system of claim 1, wherein the wearable device determines when the measurement object starts coughing before completing the inspiratory section of the respiratory cycle as a first disease,
determines when the measurement object starts coughing with the start of the expiratory section after completing the inspiratory section of the respiratory cycle as a second disease, determines when the measurement object starts coughing in the middle of the expiratory section of the respiratory cycle as a third disease, and determines when the measurement object starts coughing at the end of the expiratory section of the respiratory cycle as a fourth disease.

3. The respiration-associated coughing evaluation system of claim 1, wherein when determining the respiratory information about the measurement object, the wearable device determines the respiratory information by further considering at least one of a frequency of coughing per predetermined unit time, a ratio of an amount inhaled to an amount of air exhaled during coughing, a peak pressure of coughing, and a ratio of coughing having a predetermined intensity or more to total coughing.

4. The respiration-associated coughing evaluation system of claim 1, wherein the wearable device includes a main body including a controller determining the respiratory information about the measurement object based on the time when the measurement object starts coughing in the respiratory cycle and a communication unit transmitting the respiratory information, a band connected to the main body and detecting the respiration of the measurement object, and a neck connection microphone detecting a sound made in or around a neck of the measurement object to obtain the voice data of the measurement object, and wherein the controller of the main body obtains the respiratory cycle based on the respiration of the measurement object detected by the band and classifies the coughing using the voice data of the measurement object.

5. The respiration-associated coughing evaluation system of claim 4, wherein the wearable device further includes a chest microphone detecting at least one of whether there is a pulmonary disease, crackle, or wheezing or installed on a chest of the measurement object or on a front or rear surface around the chest.

6. The respiration-associated coughing evaluation system of claim 5, wherein the band includes a chest band detecting the respiration through the chest and an abdominal band detecting the respiration through an abdomen.

7. The respiration-associated coughing evaluation system of claim 6, wherein the neck connection microphone and the chest microphone have a concave center portion in a front surface.

8. The respiration-associated coughing evaluation system of claim 7, wherein the wearable device further includes a mask including a flow rate/flow velocity sensor electrically or communicatively connected to the main body and measuring a flow rate or flow velocity of the respiration on an inner surface.

9. A wearable device, comprising:
a main body including a controller detecting respiration of a measurement object to obtain a respiratory cycle, dividing coughing using voice data of the measurement object, and determining respiratory information about the measurement object based on a time when the measurement object starts coughing in the respiratory cycle through a relationship between the respiratory cycle of the measurement object and a start point of the coughing, and a communication unit transmitting the respiratory information;
a band connected to the main body and detecting the respiration of the measurement object; and
a neck connection microphone detecting a sound made in or around a neck of the measurement object to obtain the voice data of the measurement object, wherein the respiratory cycle is divided into an inspiratory section and an expiratory section, and wherein when determining the respiratory information about the measurement object, the controller of the main body determines whether the measurement object starts coughing before completing the inspiratory section of the respiratory cycle, whether the measurement object starts coughing with a start of the expiratory section after completing the inspiratory section of the respiratory cycle, whether the measurement object starts coughing in a middle of the expiratory section of the respiratory cycle, and whether the measurement object starts coughing at an end of the expiratory section of the respiratory cycle.

10. The wearable device of claim 9, wherein the controller of the main body determines when the measurement object starts coughing before completing the inspiratory section of the respiratory cycle as a first disease, determines when the measurement object starts coughing with the start of the expiratory section after completing the inspiratory section of the respiratory cycle as a second disease, determines when the measurement object starts coughing in the middle of the expiratory section of the respiratory cycle as a third disease, and determines when the measurement object starts coughing at the end of the expiratory section of the respiratory cycle as a fourth disease.

11. The wearable device of claim 9, wherein when determining the respiratory information about the measurement object, the controller of the main body determines the respiratory information by further considering at least one of a frequency of coughing per predetermined unit time, a ratio of an amount inhaled to an amount of air exhaled during coughing, a peak pressure of coughing, and a ratio of coughing having a predetermined intensity or more to total coughing.

12. The wearable device of claim 9, further comprising a chest microphone detecting at least one of whether there is a pulmonary disease, crackle, or wheezing or installed on a chest of the measurement object or on a front or rear surface around the chest.

13. The wearable device of claim 12, wherein the band includes a chest band detecting the respiration through the chest and an abdominal band detecting the respiration through an abdomen.

14. The wearable device of claim 13, wherein the neck connection microphone and the chest microphone have a concave center portion in a front surface.

15. The wearable device of claim 14, further comprising a mask including a flow rate/flow velocity sensor electrically or communicatively connected to the main body and measuring a flow rate or flow velocity of the respiration on an inner surface.

16. A respiration-associated coughing evaluation system, comprising:
a wearable device transmitting a respiratory cycle and voice data of a measurement object obtained by detecting respiration of the measurement object;
a server dividing coughing using the voice data of the measurement object, determining respiratory information about the measurement object based on a time when the measurement object starts coughing in the respiratory cycle through a relationship between the respiratory cycle of the measurement object and a start point of the coughing, and transmitting the respiratory information; and
a terminal receiving and displaying the respiratory information transmitted from the server, wherein the respiratory cycle is divided into an inspiratory section and an expiratory section, and wherein when determining the respiratory information about the measurement object, the server determines whether the measurement object starts coughing before completing the inspiratory section of the respiratory cycle, whether the measurement object starts coughing with a start of the expiratory section after completing the inspiratory section of the respiratory cycle, whether the measurement object starts coughing in a middle of the expiratory section of the respiratory cycle, and whether the measurement object starts coughing at an end of the expiratory section of the respiratory cycle.

17. A respiration-associated coughing evaluation method, comprising:
determining respiratory information about a measurement object based on a time when the measurement object starts coughing in an inspiratory section and an expiratory section; and
storing or transmitting the respiratory information, wherein the respiratory cycle is divided into an inspiratory section and an expiratory section, and wherein when determining the respiratory information about the measurement object, determining the respiratory information determines whether the measurement object starts coughing before completing the inspiratory section of the respiratory cycle, whether the measurement object starts coughing with a start of the expiratory section after completing the inspiratory section of the respiratory cycle, whether the measurement object starts coughing in a middle of the expiratory section of the respiratory cycle, and whether the measurement object starts coughing at an end of the expiratory section of the respiratory cycle.

18. The respiration-associated coughing evaluation method of claim 17, wherein determining the respiratory information determines the respiratory information by further considering at least one of a frequency of coughing per predetermined unit time, a ratio of an amount inhaled to an amount of air exhaled during coughing, a peak pressure of coughing, and a ratio of coughing having a predetermined intensity or more to total coughing.
